(19) United States
(12) Patent Application Publication
(10) Pub. No.: US 2005/0220812 A1
(43) Pub. Date: Oct. 6, 2005

(54) Screening process

(76) Inventors: Richard William Titball, Wiltshire (GB); Carl Nicholas Mayers, Wiltshire (GB); Melanie Lorraine Duffield, Wiltshire (GB); Sonya Claire Rowe, Wiltshire (GB); Julie Miller, Wiltshire (GB); Bryan Lingard, Wiltshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/505,809

(22) PCT Filed: Feb. 25, 2003
(86) PCT No.: PCT/GB03/00796

§ 371 Date: May 12, 2005

(30) Foreign Application Priority Data
Feb. 26, 2002 (GB) 0204387.5

(51) Int. Cl. C12Q 1/70; G01N 33/554; G01N 33/569; A61K 39/12; A61K 39/02

(52) U.S. Cl. 424/204.1; 435/5; 435/7.32; 424/234.1

(57) ABSTRACT

A method for identifying a vaccine candidate, said method comprising selecting a protein from the proteome of a target organism on the basis of a property selected from a biophysical property or the amino acid composition of that protein. For example, using this method a first set of data for a said property of a one or more vaccine antigens of a particular genus is collected, and a control set of data for said property of one or more random proteins from the same genus is also collected. These datasets are then compared and statistical differences noted. The proteome of a target species may then be examined and one or more vaccine candidates selected from that proteome on the basis that they have a property more similar to that of the first set of data

The present invention relates to a method for identifying vaccine candidates for example from the proteome of a pathogenic organism and in particular a bacteria, to vaccines identified using this method and to computer readable mediums which are useful in it.

During the past 200 years the use of vaccines to control infectious diseases caused by bacterial pathogens has proven to be both effective and safe. Many of these vaccines were discovered using an empirical approach and such vaccines include live attenuated forms of bacterial pathogens, killed bacterial cells and individual components of the bacterium (sub-units). Although many bacterial vaccines are still widely used, a shift towards reliance on antibiotic therapy for the control of many other infectious diseases occurred during the latter half of the twentieth century.

The recent appearance of antibiotic resistant strains of many bacterial pathogens has prompted a resurgence of interest in the use of vaccines to prevent disease. However, many of the existing bacterial vaccines are not considered to offer appropriate levels of protection against infection. In addition, an increased awareness of the potential for transient side effects following vaccination has prompted an increased emphasis on the use of sub-unit vaccines rather than vaccines based on whole bacterial cells. Also, there are still several infectious organisms for which no effective vaccine has yet been produced.

Whilst empirical approaches to the selection of vaccine sub-units are still employed, the selection of candidate sub-units for testing is generally dependent on a significant body of background knowledge on the molecular interactions between pathogen and host. For many bacterial pathogens this information is not available. More recently, there has been an increased awareness that bioinformatic-based approaches can allow candidate protein sub-units to be selected in silico from bacterial genome sequences. These methods can be used to screen whole genomes for potential candidates far more rapidly than empirical approaches, so providing a more rapid advance towards preclinical studies with vaccines.

In general the ‘in silico’ approaches have relied on the assumption that candidate proteins will be located on the outer surface of, or exported from, the bacterium. Some workers have first identified ORFs which would encode proteins which possess a signal sequence directing export across the cytoplasmic membrane (Gomez M, et al. Infec. Immun. 2000 66: 2323-2327; Pizza M, et al, 2000). This dataset has then been screened to eliminate proteins which include transmembrane domains (Pizza et al., 2000; Gomez et al., 2000 supra.) and to include proteins which possess lipoprotein attachment sites (Gomez et al., 2000 supra; Chakravarti et al. Vaccine. 2000 19:601-612) or other motifs associated with surface anchoring (Pizza et al., 2000 supra.; Ross et al. Vaccine. 2001 19:4135-4142). Whilst these approaches have yielded novel sub-units, the predictive power of these approaches is limited both by limited knowledge of the export and protein processing pathways in different bacterial species and by limited knowledge of the molecular architecture of outer membrane proteins. In addition, it should be borne in mind that some vaccine antigens might not be located predominantly on the outer surface of the bacterium.

The genome sequences of many bacterial pathogens have now been determined or are due for completion in the next few years, and this has prompted significant work to investigate how these genome sequences can be interpreted to provide improved pretreatments or therapies for disease. Previous workers have considered the likely cellular location of vaccine antigens on the surface of the bacterium, and used algorithms which predict the cellular location to interrogate the predicted bacterial proteome for novel vaccine candidates.

Other previous methods for the prediction of vaccine candidates have included using algorithms to locate proteins with sequence similarity to known vaccines. However, such techniques would fail to predict new families of vaccine candidates. Yet further reported methods searched for tandem repeats at the 5′ end of a gene, since such repeats have been associated with some virulence genes (Hood DW, et al. Proc Natl Acad Science USA. 1996, 93:11121-11125). However, many virulence-associated genes lack such repeats and so would not be identified by this method.

Algorithms that search for signal sequences to identify secreted proteins have also been:used by many workers to identify candidate vaccine antigens (Chakravarti et al., 2001 supra, Janulczyk R and Rasmussen M. Infect. Immun. 2001 69:4019-4026). However, such programs are unable to take into account the different methods used to export proteins and the different signal sequences possessed by different bacteria. Nor do such algorithms provide 100% accuracy when predicting the cellular locality of proteins and possible candidates may be missed. As has been previously pointed out (Montgomery D L. Brief. Bioinform. 2000 1:289-296), protein antigens having no classic leader sequence would not be identified using this method. One such example is the vaccine antigen ESAT-6 from Mycobacterium tuberculosis, a known T-cell antigen (Sonrenson A L, et al., Infect. Immun 1995 63:1710-1717, Li Z, et al, Infect. Immun. 1999 67:4780-4786, Olsen A W, et al., Infect. Immun. 2001 69:2773-2778), which would be missed using this method.

The applicants have surprisingly found that certain properties of reported protein vaccine antigens are significantly different from a representative control protein dataset. This indicates that likely vaccine antigens can be identified by comparing those properties of known protein vaccine antigens with those of randomly selected but representative proteins in a control dataset.

The present invention provides a method for identifying a vaccine candidate, said method comprising selecting a protein from the proteome of a target organism on the basis of a property selected from a biophysical property or the amino acid composition of that protein.

In particular the method requires that an algorithm is constructed based upon a comparison of the above-mentioned property of a range of proteins known to have the desired protective immunogenic property (i.e. vaccine antigens) as compared to that property of a random selection of proteins.

The term “biophysical property”, used herein refers to a bulk property of the protein as a whole, such as molecular weight or isoelectric point (pI). It has also been found that amino acid composition can act as a basis of the selection, either by considering the properties of the individual amino acids within the sequence, such as hydrophobicity, bulkiness, flexibility and mutability, and more particularly, the simple amino acid makeup or composition itself.

Surprisingly, it has been found that there is a particularly good correlation between these properties and ability of the protein to produce a protective immune response and therefore have application as a vaccine. No such correlation between such basic properties and function or activity has previously been noted.

In particular the method comprises collecting a first set of data for a said property of a one or more vaccine antigens of a particular genus, collecting a control set of data for said property of one or more random proteins from the same genus, comparing said data, examining the said property of proteins from the proteome of a target species, and selecting a vaccine candidate from that proteome which has a property more similar to that of the first set of data.

Suitably the first and control sets of data are each obtained from a plurality of proteins, which are themselves suitably obtained from a plurality of species of the selected genus.

The method may be applied to any genus of organism for which vaccines are required, for example, bacteria including mycoplasma, viruses, yeasts and bacteria, but is preferably applied to bacteria, including both gram negative and gram positive bacteria.

A list of suitable bacteria from which the datasets are constructed is set out in Table 1 hereinafter. Preferably, the datasets are constructed using proteins from all of the bacterial species listed in Table 1.

In a particularly preferred embodiment, the datasets are interrogated or analysed on the basis of the percentage composition of individual amino acids.

This embodiment therefore comprises a process which comprises the steps of analysing the individual amino acid content of proteins from one or more species having a known vaccine effect, and comparing this with the individual amino acid content of a range of randomly selected proteins from said species, and comparing the results.

A suitable comparison is carried out by first ascribing an amino acid score to each amino acid within the protein sequence using the equation:

$$Amino acid score = \frac{\begin{matrix}Percentage composition \\ vaccine antigen database\end{matrix} - \begin{matrix}Percentage composition \\ of control database\end{matrix}}{\begin{matrix}Percentage composition of \\ control database / 10\end{matrix}}$$

When this analysis is applied to all proteins derived from all the species listed in Table 1 hereinafter, each amino acid has a score shown in Table 4 hereinafter. With this information, the sequence of proteins within a proteome of a target organism can be given a “total” score, based upon applying the appropriate figure. For vaccine use, it has been found that the protein preferably scores highly on this scale. Thus for example, proteins from said target organism which are in the highest 20% of scores, suitably in the top 10%, and more preferably in the top 3% may be selected as vaccine candidates.

If required, analysis using one or more different properties can be applied in order to select a vaccine candidate with “fits” the vaccine profile more closely. In all cases, the analysis is suitably effected in silico and may be carried out using software which is in the public domain, as illustrated below.

Once the vaccine candidate has been identified, it may then be obtained and tested to establish its suitability as a vaccine. For example, it may be isolated from the bacterial source, or synthesized, for example chemically using peptide or protein synthesizer, or using recombinant DNA technology as is well known in the art. Thus a nucleotide sequence encoding the protein is incorporated into an expression vector including the necessary control elements such as a promoter, which is used to transform a host cell, which may be a prokaryotic or eukaryotic cell, but is preferably a prokaryotic host cell such as E. coli.

It may then be tested either in vitro, and/or in vivo for example in animal models and in clinical trials, to establish that it produces a protective immune response.

Vaccine candidates identified as described above form a further aspect of the invention.

In addition, vaccines which use these candidates or protective variants thereof or protective fragments of any of these, as active components, and which may include pharmaceutically acceptable carriers, as understood in the art, form a further aspect of the invention. Vaccines may be suitable for administration by various routes including oral, parenteral, inhalation, insufflation or intranasal routes, depending upon factors such as the nature of the active component and the type of formulation used. Active vaccine components may be used in the form of proteins of peptides, or nucleic acids, which encode these, may be used in such a way that they are expressed within the host animal. For example, they may be used to transform organisms such as viruses or gut colonizing organisms, which are then used as “live” vaccines, or they may be incorporated into plasmids in the form of so called “naked DNA” vaccines.

As used herein, the expression “variant” refers to sequences of amino acids which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 75% identical, and more preferably at least 90% identical to the base sequence.

Identity in this instance can be judged for example using the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The term “fragment thereof” refers to any portion of the given amino acid sequence which has the same activity as the complete amino acid sequence. Fragments will suitably comprise at least 5 and preferably at least 10 consecutive amino acids from the basic sequence.

In a further aspect, the invention provides a computer-readable medium, which contains first and control datasets, for use in the method described above, and computer readable instructions for performing the method as described above.

Newly reported vaccine antigens could be added, to further refine the positive dataset.

As described in more detail below, using the method of the invention, the applicants found that both the pI and molecular weight of the proteins in the positive dataset showed statistical significance difference from the control dataset. The two-peak pattern seen in the pI analysis occurs in all datasets tried. Bacteria are more likely to experience acidic or basic conditions in nature (and rarely encounter neutral conditions) which may account for the trough in the pI analysis at neutral conditions.

In addition, the analysis in accordance with the invention has revealed that the hydrophobicity, bulkiness, flexibility and mutability of vaccine antigens are significantly dif ferent from these properties of the control dataset. As most vaccine antigens previously described are surface exposed or secreted they are more likely to be in contact with surrounding media. This might be reflected in their hydrophobicity and may therefore explain the differences seen between the two datasets using hydrophobicity as a scale. The difference in mutability could reflect the ability of pathogens to alter their antigenic presentation and thereby evade the host's immune system. Phenotypic variation in the relevant cell-surface proteins has been seen amongst clinical isolates of some species, suggesting that antigenic proteins can mutate and evolve during the period of infection (Peterson et al, 1995). This could also account for the differences seen in the comparisons of bulkiness and flexibility since the use of small, flexible residues on a protein surface may also reflect the need for mutation.

Using the vaccine antigen amino acid scoring scale described above, it has been found that vaccine antigens have a significant scoring similarity to outer membrane and secreted proteins. Since most vaccines antigens identified to date are known to be surface exposed or secreted, this is expected. This particular scoring algorithm was able to rank known antigens within the top 10% of proteins from the Streptococcus pneumoniae proteome.

Other bacterial proteomes have also been ranked using the scoring algorithm described herein and the known vaccines antigens that are included in our positive dataset most frequently occur in the top 10% of scores (data not shown).

This study demonstrates the effective use of certain properties, in particular amino acid composition, as a tool for the prediction of vaccine candidates. The approach described here would be applicable to any pathogenic organism, and in particular bacteria, for which a proteome or a substantial part of the proteome is or becomes available. Since it does not rely on sequence similarity, motifs or sub-cellular location, it should identify vaccine candidates that other prediction tools may miss.

The method of the invention appears robust in that it allows potential vaccine candidates to be identified irrespective of the cellular location. It does not require that-a specific sequence or motif is present in the protein. For instance, using a method of the invention based upon the amino acid composition, the ESAT-6 from Mycobacterium tuberculosis, the known T-cell antigen discussed above, was the 85^{th} ranked protein in the entire predicted proteome of M. tuberculosis (i.e. in the top 3%, data not shown).

The invention will now be particularly described by way of example with reference to the accompanying tables and drawings in which:

Table 1 lists the data sources of proteins used to construct the vaccine antigen dataset. Vaccine antigen proteins were selected from the references indicated in the table.

Table 2 lists the data sources of proteins used to construct the control dataset. Proteins were selected from existing databases as shown in the table. (¹ http://www.ncbi.nlm.nih.gov; 2 http://www.sanger.ac.uk; ³ http://www.tigr.org; ⁴ http://www.genomecorp.com; ⁵ http://genome.wisc.edu; ⁶ http://www.genome.ou.edu)

Table 3 is a summary of bacterial subcellular location protein database. Proteins were selected from the SWISSPROT annotated protein database from the species listed in the table. Proteins from each subcellular location were grouped to form subcellular location databases.

Table 4 shows amino acid composition of vaccine antigen and control databases, and the results of the application of an algorithm of a preferred embodiment of the invention to them. The mean percentage amino acid composition and standard deviation of the proteins within the vaccine antigen and control databases are listed. The probability (P) of the two databases sharing the same median has been calculated by the Wilcoxon Rank Sum test and is given to three decimal places. Values of P below 0.05 are significantly different and have been allocated a score as indicated in the methods.

Table 5 shows proteins of Streptococcus pneumoniae R6 scored by 30 the vaccine antigen scale. The top 50 ranked proteins of Streptococcus pneumonia as scored by the vaccine antigen scale are listed. Other known vaccine antigens of S. pneumoniae are also shown, along with their rankings and vaccine antigen scores. *-represents vaccine candidates as previously recognised by bioinformatic methods (Hoskins et al, 2001).

Table 6 shows P scores for comparisons of positive and control atasets with databases for various sub-cellular locations. he vaccine antigen scale was used to score proteins from either the positive or control datasets and compared to databases of proteins from various cellular locations. The probability (P) of the two databases sharing the same median has been calculated by the Wilcoxon Rank Sum test.

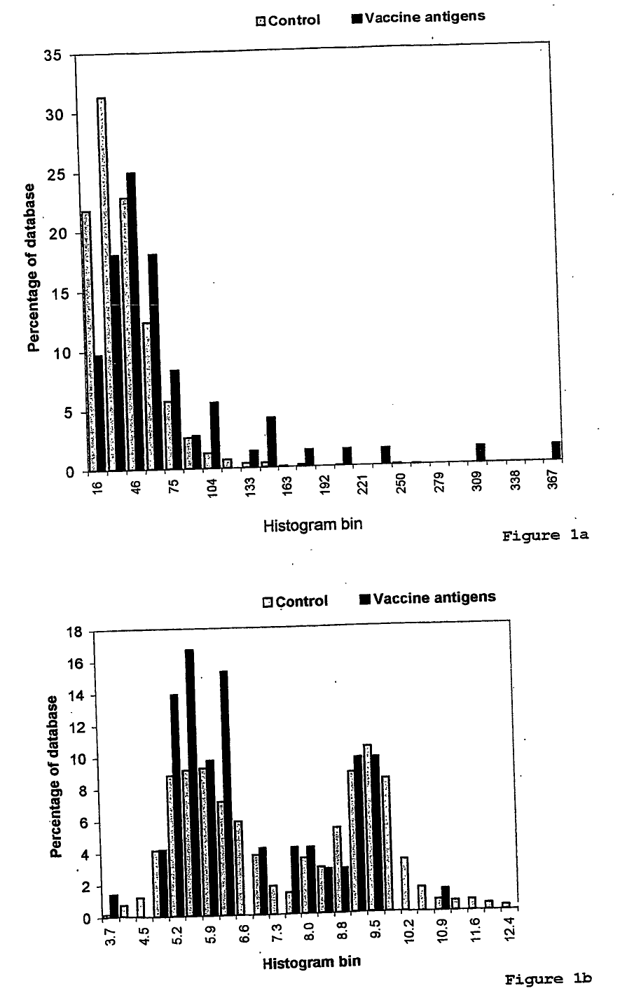

FIG. 1 shows a histogram of vaccine antigen and control databases scored by predicted molecular weight and pI. Histograms are shown of the scores obtained by analysing the vaccine antigen and control databases for: (a) predicted molecular weight and (b) predicted pI. The combined distributions for each pair of values were divided into 25 equally sized histogram bins with the x-axis labels showing the upper limit of the histogram bin. The percentage of each database within each histogram bin is shown on the y-axis.

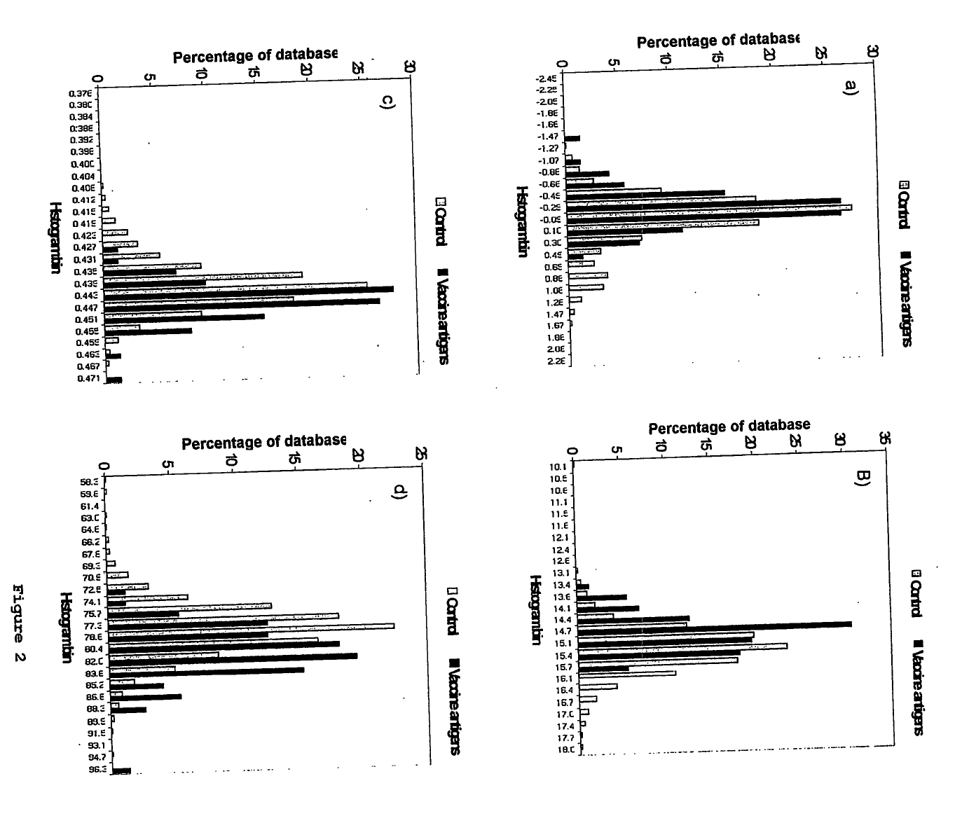

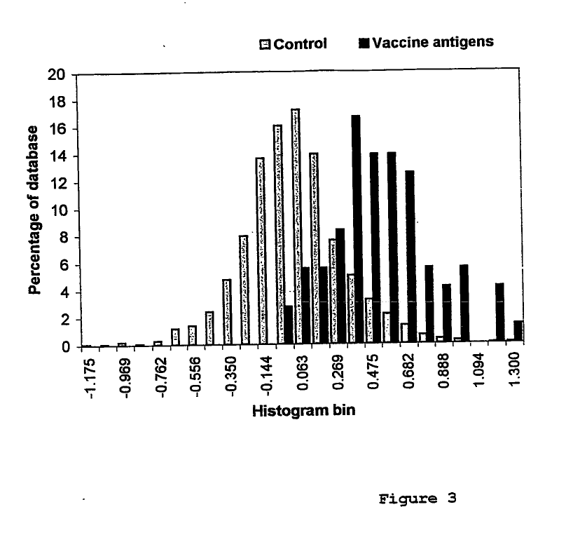

FIG. 2 shows histograms of vaccine antigen and control databases scored by four different scales. Histograms are shown of the scores obtained by scoring the vaccine antigen and control databases with: (a) Kyte-Doolittle hydrophobicity scale, (b) Zimmermann et al. bulkiness scale, (c) Bhaskaran and Ponnuswamy flexibility scale and (d) Dayhoff et al. relative mutability scale. The combined distributions for each pair of scores were divided into 25 equally sized histogram bins with the x-axis labels showing the upper limit of the histogram bin. The percentage of each database scoring a particular score is shown on the y-axis.

FIG. 3 is a histogram showing vaccine antigen and control databases scored by vaccine antigen scale. A histogram is shown of the scores obtained by scoring the vaccine antigen and control databases with the vaccine antigen scale. The percentage of each database scoring a particular score is shown on the y-axis. The combined distribution of the two populations of scores was divided into 25 equally sized histogram bins (score of 0.103 per bin), with the x-axis labels showing the upper limit of the histogram bin.

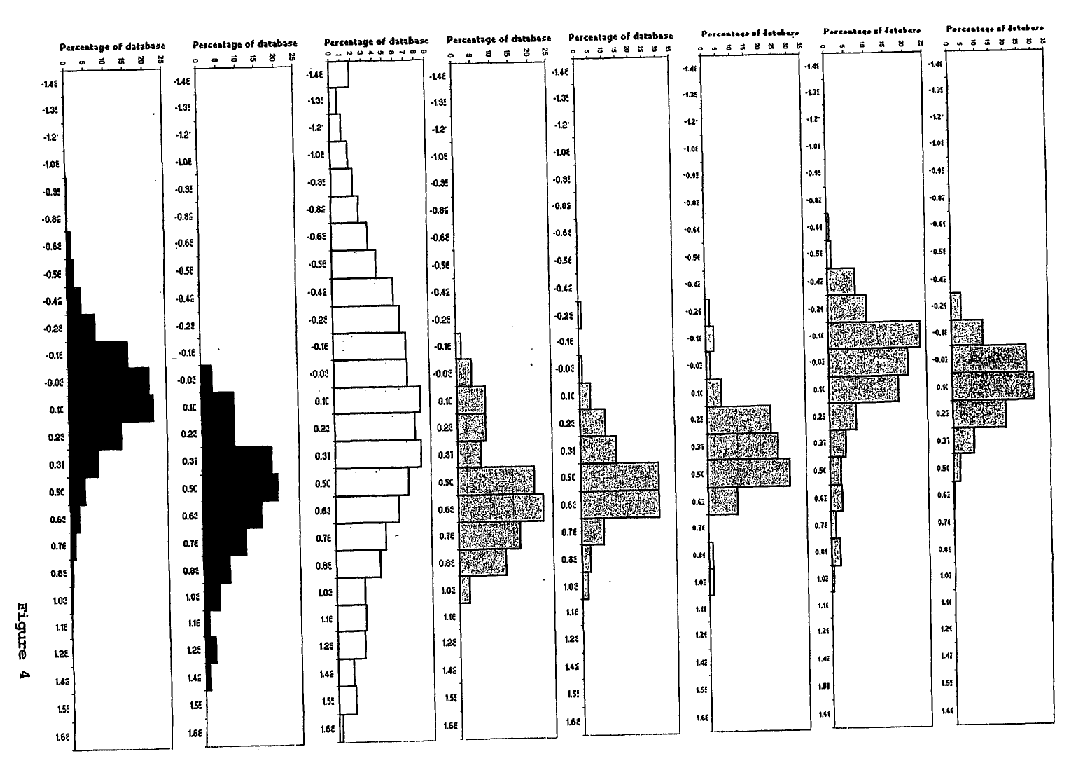

FIG. 4 shows histograms of other databases scored by the vaccine antigen scale. Histograms are shown of the scores obtained by using the vaccine antigen scale to score (a) cytoplasmic proteins, (b) inner membrane proteins, (c) periplasmic proteins, (d) outer membrane proteins, (e) secreted proteins, (f) the vaccine antigen database and (g) the control database. The percentage of each database scoring a particular score is shown on the y-axis. The combined distribution of the populations of scores was divided into 25 equally sized histogram bins, with the x-axis labels showing the upper limit of the histogram bin.

### EXAMPLE 1

### Construction of Datasets

Construction of Vaccine Antigen Dataset

Vaccine antigens were identified by patent and open literature searches to derive a list of bacterial proteins which have been shown to induce a protective response when used as immunogens in an appropriate animal model of disease. To qualify for inclusion into the database the candidate, whole or part of the protein or corresponding DNA must have been shown to induce a protective response after immunisation using an appropriate animal model of infection, or to induce a protective response against the effects of a toxic component challenge. Those chosen were entered into a FASTA formatted database file.

In total, 72 vaccine antigens were identified (Table 1). These proteins originated from 32 bacterial species in 23 genera. of the 72 antigens held within the vaccine antigen dataset, 26 originated from Gram-positive bacteria and 46 from Gram-negative bacteria (for the purposes of this study Mycobacteria were treated as Gram-positive bacteria).

The amino acid sequences of the vaccine antigens were obtained from publicly available sequence databases, primarily the NCBI database, which may be interrogated at http://www.ncbi.nlm.nih.gov. The vaccine antigen proteins identified for use in this study are shown in Table 1.

Construction of Control Dataset

In order to allow meaningful comparisons, a control database was constructed that mirrored the vaccine antigen dataset with respect to the proportion of entries from each genus. For the control dataset a single species which was considered to be representative of each genus included in the vaccine antigen dataset was selected. The species was also selected on the basis of availability of an entire predicted proteome or genome sequence. Then, for each entry in the vaccine antigen dataset, we randomly selected 35 proteins from the proteome of the corresponding species, for inclusion in the control dataset, using a routine written in PERL. In cases where a genome sequence was available but had not been annotated, the proteome was predicted using Glimmer (Delcher et al., 1999). In these cases the program fastablast.pl from TIGR (which may be found at http://www.tigr.org.uk) was adapted and used to produce a FASTA file of all the predicted protein sequences. Where no completed genome sequence was available for any member of the genus represented in the vaccine antigen dataset, all of the known proteins from the chosen species were downloaded from the publicly available protein sequence databases (NCBI). All proteome data was stored in FASTA format. The genus, species and data sources used to construct the control database are shown in Table 2.

The size of the control dataset was constructed to ensure that the final size was approximately equal to the number of proteins encoded by a typical bacterial genome. Annotated genome sequences contain protein sequences, inclusive of any signal peptides. Since the proteins in the control dataset were derived mainly from predicted proteomic and genomic data, they are inclusive of any signal sequences. To ensure that the positive database mirrored the control dataset, the sequences used were also inclusive of any signal sequences. The vaccine antigen and control datasets were used for all of the comparisons detailed below.

### EXAMPLE 2

### Analysis of Physical Properties of Proteins in the Control and Vaccine Antigen Databases

Programs were written in PERL to calculate the predicted molecular weight and predicted isoelectric point (pI) of each protein within the control and vaccine antigen databases. The results were ranked, grouped into histogram bins corresponding to increments of 15Da (FIG. 1a) or 0.4 pI units (FIG. 1b) and measured against the percentage of each database within each histogram bin. The distribution of molecular weight and pI in the two databases is shown in the histograms in FIG. 1. The statistical significance of any differences in molecular weight, pI or score was calculated by the Wilcoxon Rank Sum test (Wilcoxon, 1945; Mann & Whitney, 1947). This non-parametric test makes no assumption as to the distribution when comparing two datasets, and returns the probability of the distribution of the scores in the two databases (P score) as being identical. A P score of <0.05 was considered to be significant.

The two-peak distribution of pI values in both the control and positive datasets was also seen with all of the predicted proteomes analysed (including E. coli, M. tuberculosis, H. pylori, N. meningtidis and S. pneumoniae—data not shown). The mean values for each dataset was calculated, and to allow a comparison of the distribution of the data, the Wilcoxon Rank Sum test was applied. A comparison of positive and control datasets revealed that the distribution of molecular weight and pI values was significantly different (P=0.5×10⁻⁶ for molecular weight and P=0.002 for pI).

### EXAMPLE 3

### Amino Acid Composition of Vaccine Antigen and Control Datasets

A PERL program was written to allow each protein in the control and vaccine antigen databases to be scored according to published scales. The amino acid compositions of the proteins in the vaccine antigen and control datasets were analysed using four different scales. The total amino acids which were present in these datasets were scored for hydrophobicity (Kyte & Doolittle, 1982), flexibility (Bhaskaran & Ponnuswamy, 1988), bulkiness (Zimmermann et al., 1968) or relative mutability (Dayhoff et al., 1978) according to previously reported scoring methodologies.

The output from each of these analyses was again ranked, grouped into 25 equally distributed histogram bins and plotted as a percentage of the total database (FIG. 2a-d). The resulting P scores comparing the positive and control datasets for each scale, were found to be statistically different (hydrophobicity, p=3.7×10⁻⁶, bulkiness, p=8×10⁻¹⁴, flexibility, p=1×10⁻⁵, mutability, p=2.2×10⁻⁹).

### EXAMPLE 4

### Calculation of Amino Acid Composition of Control and Vaccine Antigen Databases

A PERL program was written to calculate the percentage amino acid composition of every protein within a FASTA formatted database. [Previous workers have described a program, ProtLock, that uses amino acid composition to predict five, protein cellular locations using the Least Mahalanobis Distance Algorithm (Cedano et al, 1997). This method was compared to the one we have developed but not found to give any better results (data not shown).]

A novel method for the prediction of bacterial protein vaccine antigens using amino acid composition to develop a new scoring algorithm was then tried.

This allowed the average amino acid composition of each database to be calculated, in addition to the standard deviation for each amino acid. Statistical significant differences in amino, acid composition between the control and vaccine antigen databases were calculated by the Wilcoxon Rank Sum test. Amino acid composition and the significance of any differences between the two databases are shown in Table 4.

Development of Scoring Algorithms

A score table was produced for amino acids based on the amino acid composition of the control and vaccine antigen datasets. The amino acid composition of each database had been calculated as described above and statistically significant differences noted. Amino acids that showed a statistically significant. difference in occurrence in the two databases were allocated a score. Each amino acid score was calculated using the mean database scores as follows:

$$Amino acid score = \frac{\begin{matrix}Percentage composition \\ vaccine antigen database\end{matrix} - \begin{matrix}Percentage composition \\ of control database\end{matrix}}{\begin{matrix}Percentage composition of \\ control database / 10\end{matrix}}$$

Amino acids that showed an increased frequency in the vaccine antigen database when compared with the control database therefore received a positive score, while those depleted in the vaccine antigen database received a negative score. Those that showed no statistically significant difference between the two databases scored 0. The scores obtained by each amino acid are shown in Table 4.

This scoring table was then used to score individual proteins in the positive and control datasets. The mean score of a protein was calculated by adding up the scores for each amino acid in the protein and dividing by the number of amino acids in the protein. The proteins were ranked on this score and then the output was allocated into 25 equally distributed histogram bins (FIG. 3). The difference between the positive and control databases is highly significant and has a P value of 2×10⁻²⁹, a higher score than achieved with the physical properties, hydrophobicity, flexibility, mutability or bulkiness.

### EXAMPLE 5

### Testing of Scoring Algorithm of Example 4

The vaccine antigen scoring scale of Example 4 was used to score proteins from each of the sub-cellular databases described. The distributions of the scores obtained by these databases are shown in FIG. 4. The vaccine antigen scoring scale was also applied to the proteome of Streptococcus pnuemoniae strain R6 (Hoskins et al, 2001), of which the top 50 scoring proteins are listed in Table 5. The positions in this scoring list of the S. pneumoniae vaccine antigens included in the positive database were then identified. The scoring positions of five other vaccine candidates, previously identified using bioinformatic techniques for predicting proteins with secretion motifs and/or similarity to predicted virulence factors (Wizemann et al, 2001), were also checked.

### EXAMPLE 6

### Vaccine Scoring Algorithm Applied to Sub-Cellular Location Protein Databases

It was hypothesised that the differences in amino acid composition of the vaccine antigen and control datasets might reflect the differences in the likely cellular locations of vaccine antigens. To investigate this possibility, the scoring algorithm described above was applied to groups of proteins with known cellular locations (cytoplasmic, inner membrane, periplasmic, outer membrane and secreted proteins).

The SWISSPROT annotated protein database http://www.expasy.ch/sprot) was searched for proteins with a defined sub-cellular location from each of the bacterial species contained in the control dataset. Any entries where the sub-cellular location of the protein was listed as ‘putative’, ‘by similarity’ or ‘suggested’ were omitted from the databases. Separate databases were constructed for each sub-cellular location, producing cytoplasmic, inner membrane, periplasmic, outer membrane and exported protein databases. Gram-positive membrane proteins were included in the,inner membrane database.

The resulting sub-cellular location databases and the number of proteins per species are listed in Table 3.

Each dataset of different sub-cellular location was compared with both the vaccine antigen and control databases. Since most currently known vaccine antigens are either surface expressed or excreted proteins, it was expected that this analysis would reveal a similarity between the positive dataset and the databases of both the outer membrane and secreted proteins. The P scores of 0.38 and 0.30 (outer membrane and secreted proteins) confirmed this (FIG. 4 and Table 6). The control dataset showed significant differences to all the sub-cellular location datasets, confirming that it contained a good random mix of proteins from all locations.

### EXAMPLE 7

### Vaccine Scoring Algorithm Applied to a Test Proteome

To evaluate whether the algorithm of Example 4 could be used to screen an entire predicted proteome for vaccine antigens, the proteome of Streptococcus pneumoniae was analysed. When the algorithm was applied to this predicted proteome, the surface protein A (PspA), a known protective antigen (Briles et al, 2000), was identified as the 11^{th} ranked protein. other known S. pneumoniae protective antigens were found ranked within the top 190 proteins, which puts them in the top 10% of the scores (Table 5). Of the 5 proteins identified by Wisemann et al. (2001) and found to give a protective immune response in a mouse model, all but one was also found in the top 10% of proteins ranked by our scoring algorithm. Of the five, a conserved hypothetical protein with a signal peptidase II cleavage site motif identified by Wizemann et al (SP101) had the worst ranking at 347 (Table 5).

### REFERENCES

Anderson G. W. et al. Infect. Immun. 1996 64 11: 4580-04585.
Bakaletz L. O., et al. Infect. Immun. 1999 67:2746-2762.
Bennett A. M., et al. Viral Immunology 1999 12:97-105.
Bhaskaram R. et al. Int. J. Pept. Protein. Res. 1988 32:242-255
Blander S. J., et al. J. Clin.Invest, 1993 91: 717-723.
Blander S. J., et al. The Journal of Immunology 1991 147:285-291.
Bolduc G. R., et al. Infect. Immun. 2000 68:4505-4517.
Borenstein L. A., et al. J Immunology 1988 140:2415:2421.
Bowden R. A., et al., J. Medical Microbiology 1998 47:39-48.
Briles D. E., et al., Infect. Immun. 2000 68:796-800.
Brodeur B. R., et al. Infect. Immun. 2000 68:5610-5618.
Brunham R. C. U.S. Pat. No. 6,235,290, 2001.
Cameron C. E., et al., Infect. Immun. 1998 66:5763-5770.
Cedano J., et al. J Mol Biol. 1997 266:594-600.
Centurion-Lara A., et al. J Experimental Medicine 1999 189:647-656.
Chakravarti D. N., et al, Vaccine. 2000 19:601-612.
Dayhoff, M. O., et al. 1978 In “Atlas of protein sequence and Structure”, Vol 5, Suppl. 3
Delcher, A. L., et al. Nuc. Acid Res. 1999 27: 4636-4641.
DeMaria T. F., et al. Infect Immun. 1996 64:5187-5192.

Denis-Mize K. S, et al. FEMS Immunology and Medical Microbiology. 2000 27:147-154.
Diaz-Montero C. M., et al. American Journal of Tropical Medical Hygene. 2001 65:371-378.
Dunkley M. L., et al. FEMS Immunology and Medical Microbiology 1999 24:221-225.
Exner M. M., et al. Infect. Immun. 2000 68:2647-2654.
Ferrero R. L., et al. Proc. Natl. Acad. Sci. USA. 1995 92:6499-6503.
Foged N. T., et al. U.S. Pat. No. 6,110,470 2000.
Ghiara P., et al. Infect. Immun. 1997 65:4996-5002.
Gilleland H. E., et al. Infect. Immun. 1988 56:1017-1022.
Gomez M., et al. Infec. Immun. 2000 66: 2323-2327.
Guzman C. A., et al. Journal of Infectious Diseases. 1999 179:901-906.
Hanson M. S., et al. Infect. Immunol. 2000 68:6457-6460.
Hanson M. S., et al. Infect Immun. 1998 66:2143-2153.
Harari I., et al. Molecular Immunology 1990 27:613-621.
Harty J. T., et al. Journal of Immunology 1995 154: 4642-4650.
Heath et al. Vaccine 1998 16:1131-1137.
Hodgson A. L., et al. Infect. and Immun. 1994 62:5275-5280.
Holder I. A., et al. Immun. 2001 69:5908-5910.
Hood D. W., et al. Proc Natl Acad Science USA. 1996. 93:11121-11125.
Hoskins J., et al. J Bacteriol. 2001 183:5709-5717.
Hotomi M., et al. Vaccine 1998 16:1950-1956.
Ikushima M., et al. FEMS Immunology & Medical Microbiology 2000 29:15-21.
Janulczyk R., et al. Infect. Immun. 2001 69:4019-4026.
Kamath A. T., et al. Clin. Exp. Immunol. 2000 120:476-482.
Kleanthous, et al. Infect Immun. 1998 66:2879-2886.
Kyd J. M., et al., Infect. Immun. 1995 63:2931-2940.
Kyte J., et al. J. Mol. Biol. 1982 157:105-132
Labandeira-Rey M., et al. Infect. Immun. 2001 69:1409-1419.
Langermann S., et al. Science 1996 276:607-611.
Lee L. H., et al. Infect. Immun. 1999 67:5799-5805.
Lee S. F., et al. Infect. Immun. 1999 67:1511-1516.
Li Z., et al. Infect. Immun. 1999 67:4780-4786.
Mamo W., et al. FEMS Immunol & Medical Microbiology. 1994 10:47-54.
Mann, H. B., et al. Ann. Math. Statist. 1947, 18:50-60
Marchetti M., et al. Vaccine 1998 16:33-37.
Marchetti M., et al. Science 1995 267:1655-1658.
Martin D., et al. Journal of Experimental Medicine 1997 185:1173-1183.
Mason, et al. Vaccine 1998 16:1336-1343.
McDonald G. A., et al. Journal of Infectious Diseases 1988 1:228-231.
0Miller J., et al. Letters in Applied Microbiology 1998 25:56-60.
Montgomery D. L., Brief. Bioinform. 2000 1:289-296.
Morris S., et al. Vaccine 2000 18:2155-2163.
Nilsson I -M, et al. J.Clin. Invest. 1998 101:2640-2649.
Nilsson I -M, et al. Journal of Infectiuos Disease 1999 180:1370-1373.
Norton P. M., et al. Vaccine 1997 15:616-619.
Ogunniyi A. D., et al. Infect. Immun. 2000 68:3028-3033.
Ogunniyi A. D., et al. Infect. Immun. 2001 69:5997-6003.
Ohwada A., et al. Journal of Antimicrobial Chemotherapy 1999 44:767-774.
Oliveira S. C., et al. Vaccine 1996 14:00959-962.
Olsen A. W., et al. Immun. 2001 69:2773-2778.
Onate A. A., et al. Infect. Immun. 1999 76:986-988.
Oysten P. C. F., et al. Infect. Immun. 1995 63:563-568.
Peterson S. N., et al. Proc. Natl. Acad. Sci. USA. 1995 92:11829:11833.
Pizza M. et al. Science 2000 287:1816-1820
Porter D. C., et al. Vaccine 1997 15:257:264.
Price B. M., et al. Infect. Immun. 2001 69:3510-3515.
Probert W. S., et al. Infect. Immun. 1994 62:1920-1926.
Radcliffe F. A., et al. Infect. Immun. 1997 65:4668-4674.
Ross B. C., et al. Vaccine. 2001 19:4135-4142.
Satin B., et al. Journal of Experimental Medicine 2000 191:1467-1476.
Sauerborn M., et al. FEMS Letters 1997 155:45-54.
Santini L., et al. Science 2000 287:1816-1820
Seong S. Y., et al. Infect. Immun. 1997 65:1541-1545.
Shahin R. D., et al. Infect. Immun. 1995 63:1195-1200.
Sonrenson A. L., et al. Infect. Immun 1995 63:1710-1717.
Streatfield S. J., et al. Vaccine 2001 19:2742-2748.
Tanghe, et al. J Immunology 1999 162:1113-1119.
Uzal F. A., et al. The Vetinary Record 1998 142:772-725.
Velaz-Faircloth M., et al. Immun. 1999 67:4243-4250.
Vishwanath S., et al. Infect. Immun. 1990 58:646-653.
Weeratna R., et al. Infect. Immun. 1994 62: 3454-3462.
West D., et al. Immun. 2001. 69:1561-1567.
Wicher, et al. Infect. Immun. 1991 59:43434348.
Wilcoxon F. Biometrics 1945, 1:80-83.
Wizemann T. M., et al. Infect. Immun. 2001 69:1593-1598.
Xiong H., et al. Immunology, 1988, 94, 0001400021, -1.
Zimmermann J. M., et al. J. Theor. Biol. 1968 21:170-201

Zhang Y., et al. Infect. Immun. 2001 69:6828-3836.

TABLE 1

| Species | Antigen | Reference(s) |
|---|---|---|
| Bacillus anthracis | Protective antigen (PA) | Miller et al., 1998 |
| Bordetella pertussis | Pertussis toxin S1 subunit | Lee et al., 1999 |
| Bordetella pertussis | Filamentous haemagglutinin (FHA) | Shahin et al., 1995 |
| Bordetella pertussis | Pertactin (P69) | Shahin et al., 1995 |
| Borrelia burgdorferi | Outer surface protein A (OspA) | Probert et al., 1994 |
| Borrelia burgdorferi | Outer surface protein B (OspB) | Hanson et al., 2000 |
| | | Probert et al., 1994 |
| Borrelia burgdorferi | Outer surface protein C (OspC) | Ikushima et al., 2000 |
| | | Probert et al., 1994 |
| Borrelia burgdorferi | Virulent strain-associated | Labandeira-Rey |
| | repetitive antigen A | et al., 2001 |
| | (VraA) | |
| Borrelia burgdorferi | Outer membrane porin protein | Exner et al., 2000 |
| | (Oms66/p66) | |
| Borrelia burgdorferi | Decorin binding protein A (DbpA) | Hanson et al., 1998 |
| Brucella abortus | Cu/Zn superoxide dismutase | Onate et al., 1999 |
| Brucella abortus | 50S Ribosomal protein L7/L12 | Oliveira et al., 1996 |
| Brucella melitensis | Outer membrane protein 25(Omp25) | Bowden et al., 1998 |
| Campylobacter jejuni | Flagellin (FlaA) | Lee et al., 1999 |
| Chlamydia trachomatis | Major outer membrane protein | EP-B-192033 |
| | (MOMP) | |
| Clostridium difficile | Toxin A | Sauerborn et al., 1997 |
| Clostridium | Alpha-toxin (Phospholipase C) | Bennett et al., 1999 |
| perfringens | | |
| Clostridium | Epsilon toxoid (typeD) | Uzal et al., 1998 |
| perfringens | | |
| Clostridium tetani | Tetanus toxin | Norton et al., 1997 |
| | | Porter et al., 1997 |
| Corynebacterium | Phopholipase D | Hodgson et al., 1994 |
| pseudotuberculosis | | |
| Escherichia coli | Heat labile enterotoxin (B | Mason et al., 1998 |
| | subunit) | |
| Escherichia coli | Adhesin (FimH) | Langermann et al., 1996 |
| Haemophilus | Fimbrin (P5) | Bakaletz et al., 1999 |
| influenzae | | |
| Haemophilus | Outer membrane protein P1 | Bolduc et al., 2000 |
| influenzae | | |
| Haemophilus | Outer membrane protein P6 | DeMaria et al., 1996 |
| influenzae | | Hotomi et al., 1998 |
| | | Kyd et al., 1995 |
| Helicobacter pylori | Cytotoxin-associated | Ghiara et al., 1997 |
| | antigen(CagA) | Marchetti et al., 1998 |
| Helicobacter pylori | Heat shock protein 10 (Hsp10) | Ferrero et al., 1995 |
| Helicobacter pylori | Neutrophil activating protein A | Satin et al., 2000 |
| | (NapA) | |
| Helicobacter pylori | Citrate synthase (GltA) | Dunkley et al., 1999 |
| Helicobacter pylori | Urease (UreB) | Kleanthous et al., 1998 |
| Helicobacter pylori | Vacuolating cytotoxin (VacA) | Marchetti et al., 1995 |
| Helicobacter pylori | Catalase | Radcliffe et al., 1997 |
| Legionella | Major Secretory Protein (MSP) | Blander et al., 1991 |
| pneumophila | | |
| Legionella | Heat shock protein 60 | Blander et al., 1993 |
| pneumophila | (Hsp60/MCMP) | |
| Legionella | Outer membrane protein S (OmpS) | Weeratna et al., 1994 |
| pneumophila | | |
| Listeria | Listeriolysin-O (LLO) | Xiong et al., 1988 |
| monocytogenes | | |
| Listeria | Major extracellular protein | Harty et al., 1995 |
| monocytogenes | (P60) | |
| Mycobacterium avium | 65 KDa Protein | Velaz-Faircloth et al., 1999 |
| Mycobacterium bovis | MPB83 | Chambers eta 1, 2000 |
| Mycobacterium bovis | Antigen 85A (Ag85A) | Velaz-Faircloth et al., 1999 |
| BCG | | |
| Mycobacterium bovis | Antigen 85B (Ag85B) | Kamath et al., 2000 |
| BCG | | |
| Mycobacterium | Phosphate transport receptor | Tanghe et al., 1999 |
| tuberculosis | PstS-3 (Ag88) | |
| Mycobacterium | Catalase-peroxidase (KatG) | Li et al., 1999 |
| tuberculosis | | Morris et al., 2000 |
| Mycobacterium | Antigen MPT63 | Morris et al., 2000 |
| tuberculosis | | |
| Mycobacterium | Early secretory antigen target 6 | Li et al., 1999 |
| tuberculosis | (ESAT-6) | Olsen et al., 2001 |
| Neisseria | Neisseria surface protein A | Martin et al., 1997 |
| meningitidis | (NspA) | |
| Neisseria | Transferrin Binding Protein | West et al., 2001 |
| meningitidis | (TbpA) | |
| Pasteurella multocida | Pasteurella multocida toxin | U.S. Pat. No |
| | (PMT) | |
| Pseudomonas | Outer membrane protein F (OprF) | Gilleland et al., 1988 |
| aeruginosa | | Price et al., 2001 |
| Pseudomonas | Pseudomonas exotoxin A (PEA) | Denis-Mize et al., 2000 |
| aeruginosa | | |
| Pseudomonas | PcV | Holder et al., 2001 |
| aeruginosa | | |
| Rickettsia conorii | Outer membrane protein A (OmpA) | Vishwanath et al., 1990 |
| Rickettsia rickettsii | Outer membrane protein B (OmpB) | Diaz-Montero et al., 2001 |
| Rickettsia rickettsii | Outer membrane protein A (OmpA) | McDonald et al., 19888 |
| Rickettsia | MBP-Bor56 protein | Seong et al., 1997 |
| tsutsugamushi | | |
| Shigella dysenteriae | Shiga toxin subunit B | Harari et al., 1990 |
| Staphylococcus aureus | Penicillin-binding protein | Ohwada et al., 1999 |
| | (MecA) | |
| Staphylococcus aureus | Fibrinogen binding protein | Mamo et al., 1994 |
| Staphylococcus aureus | Collagen adhesin | Nilsson et al., 1998 |
| Staphylococcus aureus | Recomb SEA lacking | Nilsson et al., 1999 |
| | superantigenic activity | |
| Streptococcus | Surface immunogenic protein | Brodeur et al., 2000 |
| agalactiae | (Sip)) | |
| Streptococcus | Pneumococcal surface protein A | Ogunniyi et al., 2000 |
| pneumoniae | (PspA) | |
| Streptococcus | PhpA | Zhang et al., 2001 |
| pneumoniae | | |
| Streptococcus | Pneumolysin | Ogunniyi et al., 2000 |
| pneumoniae | | |
| Streptococcus | Pneumococcal surface antigen A | Briles et al., 2000 |
| pneumoniae | (PsaA) | Ogunniyi et al., 2000 |
| Streptococcus | Fibronectin binding protein | Guzman et al., |
| pyogenes | (SfbI) | 1999 |
| Treponema pallidum | Glycerophosphodiester | Cameron et al., |
| | phosphodiesterase (Gpd) | 1998 |
| Treponema pallidum | Surface antigen 4D | Borenstein et |
| | | al., 1988 |
| Treponema pallidum | TmpB antigen | Wicher et al., |
| | | 1991 |
| Treponema pallidum | TprK | Centurion-Lara |
| | | et al., 1999 |
| Yersinia pestis | F1 capsule antigen | Heath et al., |
| | | 1998 |
| | | Oyston et al., |
| | | 1995 |
| Yersinia pestis | V antigen | Heath et al., |
| | | 1998 |
| | | Anderson et |
| | | al., 1996 |

TABLE 2

| Genus | Data Type and species | Data Source |
|---|---|---|
| Bacillus | Proteome of subtilis | NCBI¹ |
| Bordetella | Genome of pertussis | Sanger Centre² |
| Borrelia | Proteome of burgdorferi | TIGR³ |
| Brucella | Proteins from | NCBI |
| | melitensis | |
| Campylobacter | Proteome of jejuni | Sanger Centre |
| Chlamydia | Proteome of pneumoniae | TIGR |
| Clostridium | Genome acetobutylicum | Genome Theraputics⁴ |
| Corynebacterium | Genome of diptheriae | Sanger Centre |
| Escherichia | Proteome of coli 0157 | University of |
| | | Wisconsin⁵ |
| Haemophilus | Proteome of influenzae | NCBI |
| Helicobacter | Proteome of pylori | TIGR |
| Legionella | Proteins from | NCBI |
| | pneumophila | |
| Listeria | Proteome of | NCBI |
| | monocytogenes | |
| Neisseria | Proteome of | Sanger Centre |
| | meningitidis | |
| Pasteurella | Proteome of multocida | NCBI |
| Pseudomonas | Proteome of aeruginosa | NCBI |
| Rickettsia | Proteome of prowazekii | NCBI |
| Shigella | Proteins from sonnei | NCBI |
| Staphylococcus | Proteome of aureus | Sanger Centre |
| Streptococcus | Proteome of pyogenes | University of |
| | | Oklahoma⁶ |
| Treponema | Proteome of pallidum | TIGR |
| Yersinia | Proteome of pestis | Sanger Centre |

TABLE 3

| | Inner | Outer | | | Se- |
|---|---|---|---|---|---|
| Species | membrane | membrane | Periplasm | Cytoplasm | creted |
| Borrelia | 2 | 6 | 2 | 39 | 0 |
| burgdorferi | | | | | |
| Bacillus | 102 | — | — | 91 | 21 |
| subtilis | | | | | |
| Bordetella | 0 | 2 | 0 | 1 | 1 |
| pertusis | | | | | |
| Campylobacter | 0 | 1 | 0 | 13 | 0 |
| jejuni | | | | | |
| Chlamydia | 1 | 5 | 0 | 33 | 0 |
| pneumoniae | | | | | |
| Escherichia | 47 | 19 | 38 | 107 | 19 |
| coli | | | | | |
| Haemophilus | 6 | 19 | 7 | 81 | 10 |
| influenzae | | | | | |
| Helicobacter | 8 | 0 | 0 | 83 | 7 |
| pylori | | | | | |
| Staphylococcus | 18 | — | — | 22 | 8 |
| aureus | | | | | |
| Neisseria | 3 | 15 | 1 | 20 | 0 |
| meningitides | | | | | |
| Pasteurella | 2 | 2 | 0 | 32 | 0 |
| mulocida | | | | | |
| Pseudomonas | 14 | 13 | 17 | 25 | 4 |
| aeruginosa | | | | | |
| Rickettsia | 3 | 0 | 0 | 34 | 0 |
| prowazekii | | | | | |
| Streptococcus | 12 | — | — | 8 | 2 |
| pyogenes | | | | | |
| Treponema | 3 | 2 | 6 | 37 | 0 |
| pallidum | | | | | |
| Vibrio cholerae | 3 | 7 | 2 | 18 | 6 |
| Yersinia pestis | 2 | 6 | 3 | 2 | 2 |
| Total | 226 | 97 | 76 | 646 | 80 |

TABLE 4

| | | |
|---|---|---|
| | Vaccine | |
| | antigen | Control |
| Amino | database | database |

| acid | Mean | S.D. | Mean | S.D. | P | Score |
|---|---|---|---|---|---|---|
| A | 9.90 | 4.20 | 8.49 | 4.17 | 0.006 | 1.66 |
| C | 0.62 | 0.81 | 1.14 | 1.21 | 0.000 | −4.56 |
| D | 5.90 | 2.11 | 5.13 | 2.15 | 0.009 | 1.50 |
| E | 5.93 | 3.40 | 5.98 | 2.76 | 0.286 | 0 |
| F | 3.30 | 1.56 | 4.43 | 2.53 | 0.000 | −2.55 |
| G | 8.18 | 3.15 | 6.89 | 3.06 | 0.001 | 1.87 |
| H | 1.62 | 1.48 | 2.13 | 1.45 | 0.000 | −2.39 |
| I | 5.15 | 1.93 | 7.20 | 3.39 | 0.000 | −2.85 |
| K | 7.41 | 3.67 | 6.40 | 3.82 | 0.035 | 1.58 |
| L | 7.91 | 2.18 | 10.19 | 3.22 | 0.000 | −2.24 |
| M | 1.79 | 1.07 | 2.51 | 1.30 | 0.000 | −2.87 |
| N | 6.06 | 2.57 | 4.45 | 2.53 | 0.000 | 3.62 |
| P | 3.59 | 1.94 | 3.80 | 2.03 | 0.273 | 0 |
| Q | 3.65 | 1.74 | 3.63 | 1.94 | 0.380 | 0 |
| R | 3.19 | 1.97 | 5.19 | 3.14 | 0.000 | −3.85 |
| S | 7.03 | 2.75 | 6.27 | 2.29 | 0.028 | 1.21 |
| T | 7.15 | 3.06 | 5.03 | 2.04 | 0.000 | 4.21 |
| V | 6.81 | 2.00 | 6.85 | 2.60 | 0.3967 | 0 |
| W | 1.15 | 0.98 | 1.00 | 1.00 | 0.127 | 0 |
| Y | 3.65 | 1.89 | 3.29 | 1.87 | 0.110 | 0 |

TABLE 5

| Rank | S. pneumoniae Protein | Score |
|---|---|---|
| 1 | Hypothetical protein | 2.09 |
| 2 | Hypothetical protein | 2.08 |
| 3 | Hypothetical protein | 1.92 |
| 4 | Hypothetical protein | 1.89 |
| 5 | Hypothetical protein | 1.86 |
| 6 | Choline binding protein G | 1.84 |
| 7 | Hypothetical protein | 1.74 |
| 8 | Conserved hypothetical protein | 1.72 |
| 9 | ABC transporter substrate-binding | 1.69 |
| 10 | Hypothetical protein | 1.68 |
| 11 | Surface protein pspA precursor | 1.67 |
| 12 | Hypothetical protein | 1.66 |
| 13 | ABC transporter substrate-binding protein - | 1.64 |
| | maltose/maltodextrin | |
| 14 | 50S Ribosomal protein L21 | 1.63 |
| 15 | Conserved hypothetical protein | 1.62 |
| 16 | Conserved hypothetical protein | 1.58 |
| 17 | Hypothetical protein | 1.58 |
| 18 | Hypothetical protein | 1.58 |
| 19 | General stress protein GSP-781 | 1.56 |
| 20 | Hypothetical protein | 1.55 |
| 21 | Choline binding protein A | 1.54 |
| 22 | DNA-entry nuclease (competence-specific nuclease) | 1.53 |
| 23 | Serine protease | 1.49 |
| 24 | ABC transporter solute-binding protein - unknown | 1.48 |
| | substrate | |
| 25 | Hypothetical protein | 1.48 |
| 26 | Hypothetical protein | 1.47 |
| 27 | Conserved hypothetical protein, truncation | 1.46 |
| 28 | Conserved hypothetical protein | 1.45 |
| 29 | Hypothetical protein | 1.45 |
| 30 | Hypothetical protein | 1.44 |
| 31 | ABC transporter solute-binding protein - iron | 1.44 |
| | transport, truncation | |
| 32 | ABC transporter substrate-binding protein - | 1.41 |
| | oligopeptide transport | |
| 33 | ABC transporter substrate-binding protein - | 1.41 |
| | oligopeptide transport | |
| 34 | Choline binding protein | 1.41 |
| 35 | Conserved hypothetical protein | 1.40 |
| 36 | Proteinase maturation protein | 1.37 |
| 37 | Alkaline shock protein | 1.36 |
| 38 | Hypothetical protein | 1.35 |
| 39 | Hypothetical protein | 1.34 |
| 40 | ABC transporter substrate-binding protein - oligopeptid | 1.32 |
| | transport | |
| 41 | Conserved hypothetical protein | 1.30 |
| 42 | Conserved hypothetical protein | 1.30 |
| 43 | Conserved hypothetical protein | 1.29 |
| 44 | 50S Ribosomal protein L1 | 1.29 |
| 45 | Hypothetical protein | 1.29 |
| 46 | Hypothetical protein | 1.28 |
| 47 | ABC transporter substrate-binding protein - | 1.28 |
| | oligopeptide transport | |
| 48 | ABC transporter substrate-binding protein - sugar | 1.28 |
| | transport | |
| 49 | Hypothetical protein | 1.27 |
| 50 | Choline-binding protein F | 1.26 |

Other known vaccine antigens

| | | |
|---|---|---|
| 90 | ABC transporter substrate-binding protein - manganese | 1.02 |
| | transport. | |
| 167 | Histidine Motif-Containing protein | 0.82 |
| 169 | Pneumolysin (sulfhydryl-activated toxin that lyses | 0.82 |
| | cholesterol containing membranes) | |
| 72 | Cell wall-associated serine proteinase precursor PrtA | 1.12 |
| 91 | 1,4-beta-N-acetylmuramidase | 1.02 |
| 129 | Endo-beta-N-acetylglucosaminidase | 0.90 |
| 187 | Pneumococcal histidine triad protein A precursor | 0.78 |
| 347 | Conserved hypothetical protein | 0.49 |

TABLE 6

| | Vs Positive Dataset | Vs Control Dataset |
|---|---|---|
| Cytoplasmic | 7.7 × 10⁻³⁰ | 1.1 × 10⁻⁵ |
| Inner Membrane | 1.4 × 10⁻²³ | 1.3 × 10⁻³ |
| Periplasmic | 8.5 × 10⁻⁴ | 1.6 × 10⁻²³ |
| Outer Membrane | 0.38 | 1.5 × 10⁻⁴¹ |
| Seceted | 0.30 | 5.2 × 10⁻³³ |
| MHCPEP | 1.6 × 10⁻⁸ | 6.2 × 10⁻²² |

1. A method for identifying a vaccine candidate, said method comprising selecting a protein from the proteome of a target organism on the basis of a property, wherein the property is selected from a biophysical property or amino acid composition of said protein:

2. The method of claim 1 which comprises collecting a first set of data for said property of one or more vaccine antigens of a particular genus, collecting a control set of data for said property of one or more random proteins from the same genus, comparing said data sets, examining the property of proteins from the proteome of a target species, and selecting a vaccine candidate from the target species proteome which has a property more similar to that of the first set of data.

3. The method of claim 2 wherein the first and control sets of data are each obtained from a plurality of proteins.

4. The method of claim 3 wherein the proteins are from a plurality of species of said genus.

5. The method of claim 1 wherein the genus is bacteria, yeast or virus.

6. The method of claim 5 wherein the genus is bacteria.

7. The method of claim 6 wherein the data sets are obtained using proteins from one or more bacterial species selected from the group consisting of Bacillus anthracis, Bordetella pertussis, Borrelia burgdorferi, Brucella abortus, Brucella melitensis, Campylobacter jejuni, Chlamydia trachomatis, Clostridium difficile, Clostridium perfringens, Clostridium tetani, Corynebacterium pseudotuberculosis, Escherichia coli, Haemophilus influenzae, Helicobacter pylori, Legionella pneumophila, Listeria inonocytogenes, Mycobacterium avium, Mycobacterium bovis, Mycobacterium bovis BCG, Mycobacterium tuberculosis, Neisseria meningitides, Pasteurella multocida, Pseudomonas aeruginosa, Rickettsia conorii, Rickettsia rickettsii, Rickettsia tsutsugamushi, Shigella dysenteriae, Staphylococcus aureus, Streptococcus agalactiae, Streptococcus pneumoniae, Streptococcus pyogenes, Treponema pallidum and Yersinia pestis.

8. The method of claim 7 wherein the data sets are obtained using all of the bacterial species Bacillus anthracis, Bordetella pertussis, Borrelia burgdorferi, Brucella abortus, Brucella melitensis, Campylobacter jejuni, Chlamydia trachomatis, Clostridium difficile, Clostridium perfringens, Clostridium tetani, Corynebacterium pseudotuberculosis, Escherichia coli, Haemophilus influenzae, Helicobacter pylori, Legionella pneumophila, Listeria monocytogenes, Mycobacterium avium, Mycobacterium bovis, Mycobacterium bovis BCG, Mycobacterium tuberculosis, Neisseria meningitides, Pasteurella multocida, Pseudomonas aeruginosa, Rickettsia conorii, Rickettsia rickettsia, Rickettsia tsutsugamushi, Shigella dysenteriae, Staphylococcus aureus, Streptococcus agalactiae, Streptococcus pneumoniae, Streptococcus pyogenes, Treponema pallidum and Yersinia pestis.

9. The method of claim 1 wherein the property is a biophysical property selected from the group consisting of molecular weight and isoelectric point.

10. The method claim 1 wherein the property is amino acid composition.

11. The method of claim 10 wherein the amino acid composition is analysed on the basis of percentage composition of individual amino acids, or a property of those amino acids.

12. The method of claim 11 wherein the amino acid composition is analysed on the basis of a property of those amino acids, and wherein the property is selected from the group consisting of hydrophobicity, flexibility, bulkiness and mutability.

13. The method of claim 10 wherein the amino acid composition is analysed on the basis of percentage composition of individual amino acids.

14. The method of claim 13 wherein an amino acid score is ascribed to each individual amino acid within the protein sequence using the equation:
$$Amino acid score = \frac{\begin{matrix}Percentage composition \\ vaccine antigen database\end{matrix} - \begin{matrix}Percentage composition \\ of control database\end{matrix}}{\begin{matrix}Percentage composition of \\ control database / 10\end{matrix}}$$

15. The method of claim 10 wherein the proteins within a target organism are accorded a score based on the amino acid content, wherein each amino acid merits a score according to the following:

| | |
|---|---|
| Alanine A | 1.66 |
| Cysteine C | −4.56 |
| Aspartic acid D | 1.50 |
| Glutamic acid E | 0 |
| Phenylalanine F | −2.55 |
| Glycine G | 1.87 |
| Histidine H | −2.39 |
| Isoleucine I | −2.85 |
| Lysine K | 1.58 |
| Leucine L | −2.24 |
| Methionine M | −2.87 |
| Asparagine N | 3.62 |
| Proline P | 0 |
| Glutamine Q | 0 |
| Arginine R | −3.85 |
| Serine S | 1.21 |
| Tyrosine T | 4.21 |
| Valine V | 0 |
| Tryptophan W | 0 |
| Tyrosine Y | 0 |

and wherein, one or more proteins from said target organism which are in the highest 20% of scores are selected as vaccine candidates.

16. The method of claim 15 wherein vaccine candidates are selected from the proteins which score in the highest 10%.

17. The method of claim 1, further comprising obtaining and testing said protein as a vaccine.

18. The method of claim 2 wherein the analysis of data sets is conducted in silico.

19. A vaccine candidate identified using the method claim 1.

20. A vaccine comprising the vaccine candidate of claim 19, or a fragment or variant thereof which produces a protective immune response.

21. The vaccine of claim 20 wherein the vaccine is in the form of a protein or polypeptide.

22. The vaccine of claim 21 wherein the vaccine comprises a nucleic acid which encodes the vaccine candidate or a fragment or variant thereof which produces a protective immune response.

23. The vaccine of claim 22, wherein the vaccine is a live vaccine.

24. A computer-readable medium, which contains the first and control data sets, for use in the method according to claim 2, and computer readable instructions for performing the method according to claim 2.
